(19) United States
(12) Patent Application Publication
(10) Pub. No.: US 2025/0288435 A1
(43) Pub. Date: Sep. 18, 2025

(54) PROSTHETIC SUSPENSION SYSTEM WITH COMBINATION OF SUSPENSION VALVE AND EXPULSION VALVE

(71) Applicant: COYOTE O&P INC, BOISE, ID (US)

(72) Inventors: MATTHEW J. PERKINS, BOISE, ID (US); WILLIAM SEXTON, BOISE, ID (US); JONATHAN KLEMER, BOISE, ID (US)

(21) Appl. No.: 19/078,093

(22) Filed: Mar. 12, 2025

Related U.S. Application Data
(60) Provisional application No. 63/564,482, filed on Mar. 12, 2024.

(51) Int. Cl. A61F 2/80

(52) U.S. Cl. CPC A61F 2/80; A61F 2002/805

(57) ABSTRACT

A prosthetic dual-suspension system secures a socket-liner pin to a lock at the distal end of the socket, provides a seal that prevents air from flowing through the lock to the bottom of the socket and interfering with proper suction, and further includes a one-way air-expulsion valve in the lock that expels air from the socket, as needed during use of the prosthetic, especially during the compressive portions of the user's gait. The expulsion valve may be incorporated into the lock's spring-biased push-button system that actuates suction- and pin-release. The expulsion valve automatically opens when the socket pressure goes over the valve's pre-determined “crack” pressure.

### BACKGROUND OF THE INVENTION

This application claims benefit of U.S. Provisional Application Ser. No. 63/564,482, filed Mar. 12, 2024, the entire disclosure of which is incorporated herein by this reference.

### FIELD OF THE INVENTION

The invented technology relates to improvements in a dual-suspension system for suspending a prosthesis from a wearer's residual limb. A dual-suspension system includes a socket having a lock device that mechanically connects to the pin of a limb-liner that is worn on a residual limb, which limb-liner is adapted to seal to portions of the interior wall of the hard socket (or simply “socket”). Said dual-suspension system further includes a suspension valve adapted to prevent air from leaking through the lock device and up into the socket interior, as such leaking may interfere with proper suspension of the prosthetic on the wearer's residual limb. More specifically, the invention relates to the dual-suspension system being adapted to allow air expulsion from the interior of the socket when the socket air pressure rises above a desired amount, for example, because of air leaking into the socket between the upper end of the socket and the liner during use. Certain embodiments provide a compact and convenient expulsion valve incorporated into the lock device to provide more accurate control of the pressure in the socket for improved suspension of the prosthetic from the residual limb and more comfort for the wearer. In certain embodiments, the expulsion valve is incorporated into a unit that is used as a push-button for both suspension valve operation and pin-release operation, thus reducing or eliminating the need for expulsion valves to be provided on the wall of the socket.

### SUMMARY

A prosthetic dual-suspension system secures/connects a pin of a limb-liner installed on a residual limb to a lock device at the bottom/distal end of the socket, and also provides an airtight seal that prevents air from flowing through the lock device into the bottom of the socket so that effective suction is maintained between the lower end of the limb-liner and the socket interior surface. Thus, the combination of mechanical securement/connection of the pin to the socket, plus suction in the socket that tends to keep the limb-liner-covered limb in the socket, is called a “dual-suspension” system for a leg prosthetic. Maintaining an amount of suction that is effective for secure, reliable, and comfortable suspension can be difficult in view of the wearer's walking, running, or other motions that may push and pull the liner-covered limb relative to the socket. Such motions tend to compress air that resides or has leaked into the socket interior, typically from between the liner and the top end of the socket, and/or tend to cause even more leaking into the socket interior, both of which tend to reduce effectiveness and comfort of the suspension. The invention further improves the prosthetic dual-suspension system by providing a one-way air-expulsion valve in or on the lock device. The provided expulsion valve allows expulsion of air from the lower end of the hard socket interior when pressure in said lower end builds up above a desired level. This expulsion may be needed or desirable for suspension effectiveness and comfort during use of the prosthetic especially during the compressive portions of the user's gait. In certain embodiments, the one-way air-expulsion valve is provided in, or replaces, the lock device element that is used as a push-button (also “plunger/handle”) for both suspension valve operation and pin-release operations. Thus, certain embodiments of the invention comprise a multiple-function valve unit/assembly that can be original equipment manufacture in/on, or retrofit into/onto, a suspension lock device. The valve unit/assembly's multiple functions comprise: operation as a suspension valve to prevent air leaking through the lock into the socket interior, operation as an expulsion valve that releases air from the bottom of the socket interior upon compressive limb movement and thus preventing an uncomfortable and operationally-undesirable “pistoning” effect; and operation as a convenient actuator for liner-pin release and unsealing of the suction when desired. These multiple purposes are accomplished by a single, compact unit/assembly, wherein the expulsion function operates automatically, during use of the installed prosthetic when the suspension valve is closed and the pin is locked-in, and the push-button actuation is operated manually by wearer or technician manually to open the suspension valve and release the pin whenever the prosthetic is to be removed. These multiple functions are therefore accomplished in ways that solve some or all of previous problems and discomfort associated with suction control and pistoning inside a hard socket of a leg prosthetic, while providing for intuitive and convenient prosthetic donning and removal by the wearer or technician.

### BRIEF DESCRIPTION OF THE DRAWINGS

FIG. 1 is a perspective left-front view of one embodiment of a hard socket for a leg prosthesis, wherein the socket comprises an embodiment of the invented lock device (or “lock”) comprising one embodiment of an expulsion valve.

FIG. 2 shows the socket of FIG. 1, with a liner-covered residual limb being inserted down into the socket interior, wherein the limb-liner comprises one embodiment of a liner-pin.

FIG. 3 shows the socket of FIG. 1, wherein the liner-covered residual limb has been further inserted into the socket so that the liner pin has been pushed fully down into, and captured inside, the central axial hole of the lock, thus connecting/securing the liner to the lock and suspending the leg prosthetic from the liner-covered residual limb.

FIG. 4A is a perspective left-front view of the lock of FIG. 1, removed from the socket, but shown with the liner-pin still in the lock (without showing the liner).

FIG. 4B is a perspective left-rearview of the lock and pin of FIG. 4A.

FIG. 5A is a front view of the lock of FIG. 1.

FIG. 5B is a top view of the lock of FIG. 5A.

FIG. 5C is a left view of the lock of FIG. 5A.

FIG. 5D is a rear view of the lock of FIG. 5A.

FIG. 5E is a bottom view of the lock of FIG. 5A with the leg post portion in broken-line.

FIG. 5F is a right view of the lock of FIG. 5A.

FIG. 6 is an exploded, left-rear-side perspective view of the lock of FIGS. 4A and B including the liner-pin, showing an embodiment of the lock's multiple-functional suction control and release assembly (22) that includes sliding assembly (42) having multiple-functional valve unit/assembly (60).

FIGS. 7A and 7B are top-left (or “top-outer-end”) and bottom-right (or “bottom inner-end”) perspective views, respectively, of the locking plate of the sliding assembly of the lock of FIG. 6.

FIGS. 8A and 8B are left-end (or “outer-end”) and right-end (or “inner-end”) perspective views, respectively, of the multiple-functional valve unit/assembly of FIG. 6, also called “expulsion valve unit” due to a one-way expulsion valve being a key feature of this unit/assembly.

FIG. 9 is an exploded left-end (or “outer-end”) view of the expulsion valve unit of FIG. 6.

FIG. 10A is a side view of the expulsion valve unit of FIG. 6.

FIG. 10B is a longitudinal cross-sectional view of the expulsion valve unit of FIG. 6.

FIG. 10C is a left-end (outer-end) view of the expulsion valve unit of FIG. 6.

FIG. 10D is a right-end (inner-end) view of the expulsion valve unit of FIG. 6.

FIG. 11A is a longitudinal cross-sectional view of the expulsion valve unit of FIG. 6 in a closed condition, the cross-section being taken at 90 degrees from the cross-section in FIG. 10B for easier viewing of the expulsion valve operation.

FIG. 11B is the longitudinal cross-sectional view of the expulsion valve unit as in FIG. 11A, except that the expulsion valve is open.

FIG. 12 is a radial cross-sectional view of the lock of FIG. 1, viewed along the line 12-12 in FIG. 5D, with the expulsion valve and the suspension valve closed.

FIG. 13 is a longitudinal (axial) cross-sectional view of the lock of FIG. 1, viewed along the line 13-13 in FIGS. 5C, with the expulsion valve and the suspension valve closed, this view showing the expulsion valve cross-sectional shape (as in FIG. 11A) resulting when the expulsion valve is cross-sectioned 90 degrees relative to FIG. 12.

FIG. 14 is a cross-sectional view of the lock of FIG. 1, except that the expulsion valve has been rotated in its valve body (62) on its longitudinal axis for this view (and likewise in FIGS. 15-17) 90 degrees compared to its orientation in FIG. 13 for better viewing of the expulsion valve operation. This view portrays the embodiment after the liner-pin has been fully inserted into the lock and the installed prosthesis is ready for use. In this installed configuration prior to the wearer's compressive movement, the expulsion valve is closed, the pin-sliding assembly is biased to the left so that the suspension valve is closed, and air cannot enter the expulsion valve (see A1) or the suspension valve (see A2).

FIG. 15 is a cross-sectional view of the lock and pin of FIG. 14, wherein the wearer's movement has started, particularly the compressive portion(s) of walking, and has increased the pressure in the socket above the “crack point” or “pop point” of the expulsion valve so that the expulsion valve has opened and air is flowing out of the expulsion valve to reduce pressure in the socket.

FIG. 16 is a cross-sectional view of the lock and pin of FIGS. 14 and 15, except showing the lock elements' positions at the start of removing the prosthetic from the wearer, wherein the wearer or other person has pressed the push-button (force/movement arrows at far left) and the sliding assembly has slid to the right, including the locking plate (force/movement arrow at far right), so that the pin is no longer locked into the lock device. Also, said pushing and consequent sliding of the sliding assembly has opened/unsealed the suspension valve to allow air flow (A4) through the lock and into the socket due to the suction in the socket. Here there is no pressure/force to open the expulsion valve, so it remains closed.

FIG. 17 is a cross-sectional view of the lock and pin as in FIG. 16, except showing the result of the actions in FIG. 16, which is that the socket pressure has equalized with the environment, which tends to release the suction liner from the socket, and the wearer is pulling his/her limb, including the liner and pin, away from the socket to remove the prosthetic. Also in this view, the push-button has been released and the sliding assembly has responded to spring-bias by sliding to the left to close the suspension valve. Still, there is no pressure/force to open the expulsion valve, so it remains closed.

### DETAILED DESCRIPTION OF CERTAIN EMBODIMENTS

### General Comments:

In the Figures are shown embodiments of the invented apparatus and/or methods for improving suspension of a prosthesis from a residual limb. Certain embodiments comprise a lock device (or “lock”) in a hard socket (or “socket”) of a prosthesis, the lock having a mechanism to lock a limb-liner pin to the socket by capturing an end of the pin in the lock and a sealing mechanism to prevent air from leaking through the lock into the socket interior, and the lock further having an expulsion valve for expelling air from the socket through the lock and to the environment. Said expelling is beneficial for modulating/adjusting the pressure inside the socket (between the socket wall and the liner on the limb), as maintaining pressure at a “partial vacuum” or “suction” inside the socket is a goal/target known and understood in the prosthetics field for dual-suspension prosthetics. Compressive movement during walking/running by the wearer tends to compress what air is inside the socket, which can cause undesirable “pistoning” as is also known in this field of prosthetics. Embodiments of the present invention are adapted to solve or reduce the problems caused by the pressure fluctuation due to said compressive movement. It may be noted that preferred embodiments utilize both locking of the liner pin and modulating/adjusting of a partial-vacuum/suction between the liner and the socket, and preferably do not comprise establishing full or even near-full vacuum inside the socket and preferably do not comprise or use a vacuum pump. Also, the preferred embodiments do not comprise or use a lanyard or other strap for connecting the socket to the liner to any portion of the socket or prosthetic. Note that the limb-liner may also be called a “socket-liner” as the liner is installed over the residual limb to elastically grip the limb, but, in use, is also positioned against, and sealing with, the interior surface of the socket.

Upon buildup of pressure in the socket above the desired amount for effective suction suspension and/or for the comfort of a particular prosthetic or patient, the expulsion valve will open to allow outward air flow a sufficient amount of volume and/or time to reduce socket pressure to the desired amount. Various expulsion valves having various one-way “crack pressures” may be selected and installed, based on the amount of suction that is effective and comfortable for a particular wearer. In preferred embodiments, this “crack” or “pop” pressure is low, for example, when the pressure inside the distal region of the socket well is above the desired suction pressure, for example, 1-3 psi above the environmental/outside air pressure. This crack/pop pressure may be reached during many or all of the compression phases of the wearer's gait, when the residual limb presses further into the socket, and the resulting air expulsion through valve 80, plus the release of compression and the transition to the suspension phase of the wearer's gait, bring the socket pressure down so the socket again reaches a slightly-negative pressure, or “suction pressure”, to maintains comfortable and effective suction suspension. The elastomeric valve member 80 may exhibit a very accurate and reproducible response at the low crack pressure, and, in addition, sound dampening aid/or filtration pads may further reduce sound and fouling of the preferred electromeric valve member. Thus, preferred embodiments are designed so that the frequent valve 80 opening and closing, and air-expelling, are quiet, so the expulsion system works discreetly to maintain comfortable and effective suction suspension even when air leaks into the socket from the top of the socket.

In preferred embodiments, the one-way air-expulsion valve operates automatically without pushing/moving the push-button of the pin-lock, based on pressure inside the lower socket which is in fluid/air communication with the inner end of the expulsion valve. Therefore, in preferred embodiments, the expulsion valve is not hand-operated by a person and does not have a handle for a person to use to open or close the expulsion valve. The expulsion valve will typically open only during normal use of the prosthetic (when the suspension valve is closed), for intermittent, short amounts of time especially during periods when the user is walking, to expel small amounts of air from the socket when the pressure in the socket goes over the valve's pre-determined “crack” pressure. This is beneficial because air may slowly enter the socket over time from the top of the socket, but will be expelled through the one-way valve, to maintain hypobaric suction in the socket and, hence, maintain effective and comfortable suspension. In certain retrofit embodiments, an existing push-button of a commercial pin-lock may be replaced by the invented push-button that comprises both suspension-valve and expulsion valve features, to serve both as the push-button for the pin-lock and the suspension valve, and as the automatic expulsion valve.

### Referring Specifically to the Figures:

The dual-suspension system comprises a limb-liner pin-lock device 20 (or “suspension lock” or simply “lock”) at the bottom of the socket 10 that mechanically captures the liner-pin 26 and to which the leg prosthesis post/leg 24 and foot, blade or other foot-prosthesis (not shown) are attached. The lock 20 comprises a suspension valve to maintain suction in the socket interior between the liner L and the socket 10 by preventing air to enter the bottom of the socket 10 through the lock 20. For exemplary locks that may incorporate, or be modified for use in, certain embodiments of the invention, see for example the AIRLOCK™ dual-suspension pin system (htps://www.coyote.us/airlock) from Coyote O&P, Inc (an Idaho Corporation). Many limb-liners with lower-end threaded pins are available commercially from US or overseas prosthetics companies and technicians.

The invention relates to adding an expulsion valve to the lock, wherein the expulsion valve allows air to escape/vent from the socket, through the lock to the atmosphere, when the air pressure builds up in the socket interior, for example, due to air “leaking into the socket” over time between the liner and the interior surface of the top of the socket. While the liner may be made of elastomeric materials, such as urethane, silicone, and thermoplastic elastomer (TPE) for example, differences in a liner's chemical composition, shape, geometry, and manufacturing determine a liner's overall performance, for example affecting the “grip” of the liner on the residual limb and the “sealing” of the liner to the interior surface of the socket. Thus, air leaking into the socket from the socket top may occur due to imperfections in the seal between the liner and the interior surface of the hard socket and the movement of the liner-covered residual limb especially relative to the socket upper end during walking and other wearer actions. Typically, expulsion of air through the expulsion valve will occur during the compression phase of the wearer's gait, when the residual limb presses into the socket, whereby this expulsion of air helps maintain comfortable and effective suction suspension in spite of said air leaking into the socket interior.

The Figures show one, but not the only, embodiment of a prosthetic dual-suspension system comprising an expulsion valve 80 incorporated into a multiple-function valve assembly 60 that is incorporated into a sliding assembly 42 of the multiple-functional suction control and release assembly 22 of the lock 20. “Push-button assembly” and “push-button operation” may be a convenient term for certain of the invented apparatus and operations of assembly 22, as the wearer/user need only push outer end 70 of the valve assembly to perform the functions over which he/she has control; other functions are automatic due to the preferred design and biasing of components into default positions. The default position of the sliding assembly is with the suspension valve biased to be closed/sealed and the expulsion valve closed/sealed. The suspension valve in certain embodiments may be said to comprise, consist essentially of, or consist of a seal provided on the sliding assembly 42, preferably on multiple-function valve assembly 60, that seals against a surface of the lock such as a slanted surface, preferably of a retainer 50 that holds the sliding assembly in the lock. In certain embodiments of the multiple function valve assembly 60, the default position places the outer end 70 of the valve assembly 60 extending from the lock to be exposed outside the lock to be accessible and usable as the push-button. The multiple-function valve assembly 60 is operatively connected to the locking plate 54 of the sliding assembly 42 that captures the liner-pin when the sliding assembly is in the default position, so that pushing of the outer end 70 actuates said plate 54 to release the liner pin. Therefore, when the wearer or a technician desires to remove the leg prosthesis, the wearer/technician will press the push-button inward toward the central longitudinal axis of the lock, which disengages/releases the pin-lock and also opens the suspension valve to release the suction inside the socket. Therefore, the Figures portray how the combination of suspension valve and expulsion valve may be assembled into a single unit that is utilized in the area of, and/or in place of, a conventional push-button/plunger of the lock, resulting in the single unit serving multiple purposes: 1) allowing air-expulsion as needed for maintaining effective and comfortable suction in the prosthetic due to air leaks in between the socket and liner over time, and 2) providing the push-button/plunger to open the suspension valve, aka “break the lock's suction seal”, so air flows through the lock and into the bottom of the socket and to also unlock the liner-pin from the lock so that the wearer may remove the prosthetic from his/her residual limb.

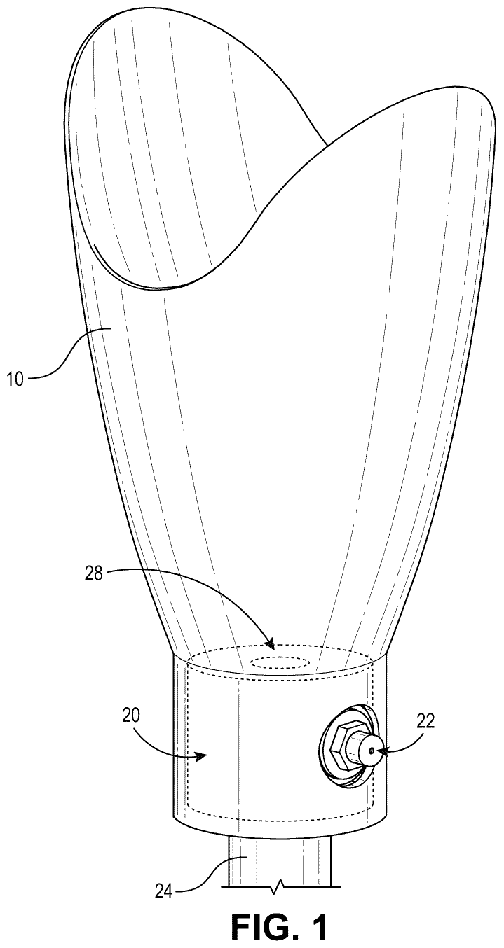

FIG. 1 shows an exemplary hard socket 10 for receiving a residual limb, preferably in a limb liner L (or “socket liner”). FIG. 1 is a perspective left-front view of one embodiment of a socket for a leg prosthesis, wherein the socket comprises a lock device (or “lock”) 20 (in dashed lines in FIG. 1) substantially enclosed by the sidewall and bottom wall of the socket but with a portion of the lock protrudes out through the hard socket sidewall near the bottom of the socket for access by a wearer. In FIG. 1, only the outer push-button end protrudes out from the socket to be accessible by a person, but retainer 50 may be removed to slide out or otherwise access the sliding assembly 42 for cleaning and maintenance.

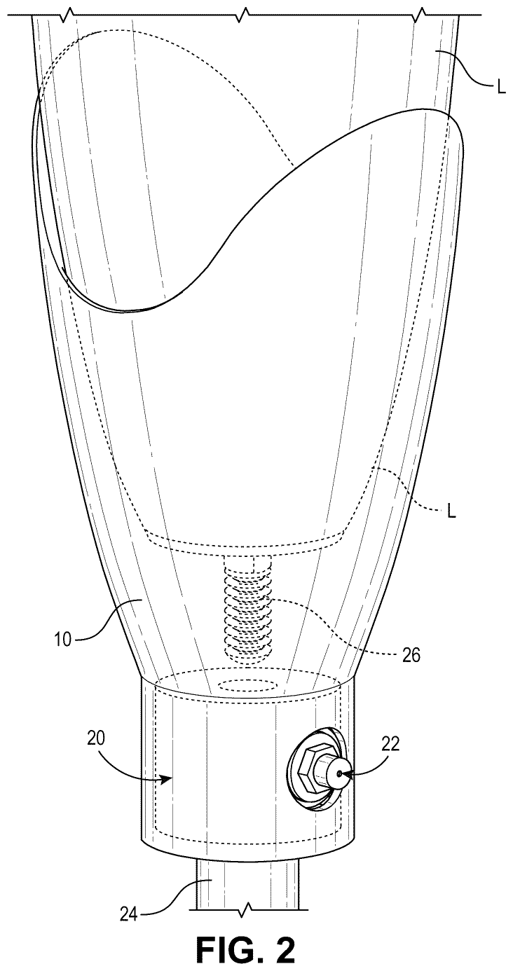

FIG. 2 shows the socket of FIG. 1, with a liner-covered residual limb being inserted into the socket interior toward lock 20. Limb-liner L comprises one embodiment of a liner-pin 26.

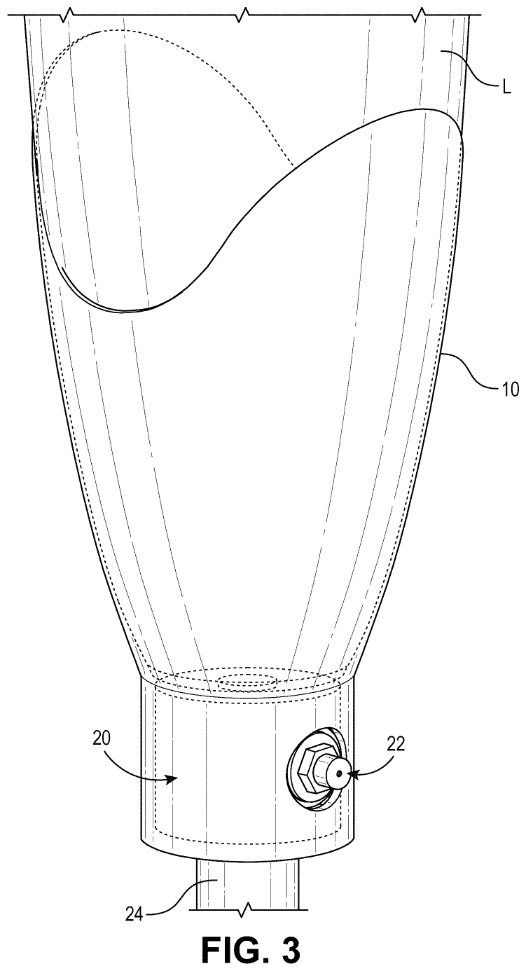

FIG. 3 shows the socket 10, wherein the liner-covered residual limb has been further inserted into the socket to an extent that the liner pin 26 is pushed fully down into, and captured inside, the central axial hole (28) of the lock, thus connecting/securing the liner L to the lock 20 and mechanically suspending the leg prosthesis from the liner-covered residual limb.

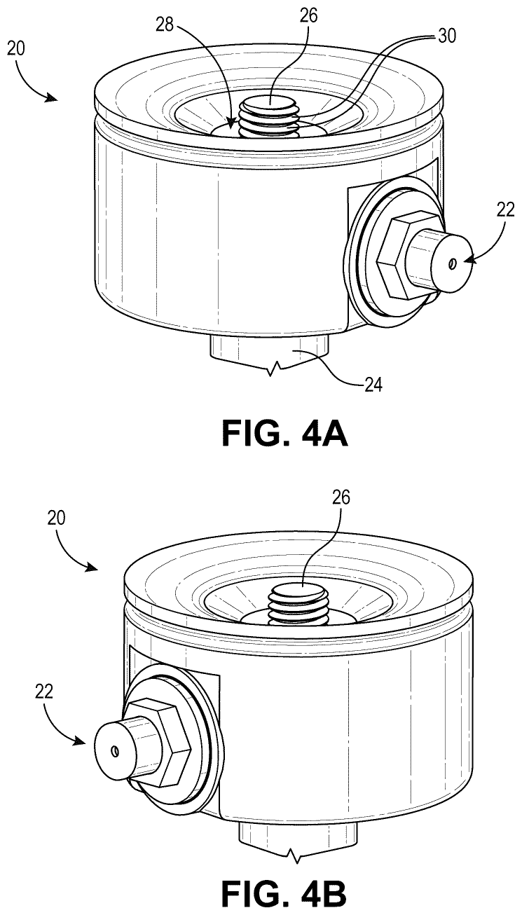

FIG. 4A is a perspective left-front view of the lock 20 of FIG. 1, removed from the socket 10, but shown with the liner-pin 26 connected/secured in the central axial hole 28 of the lock 20 (without showing the liner L). The pin 26 comprises threads, and recesses 30 between the threads, that are important in the capture of the pin 26 to secure it to the lock 20. FIG. 4B is a perspective left-rearview of the lock and pin of FIG. 4A.

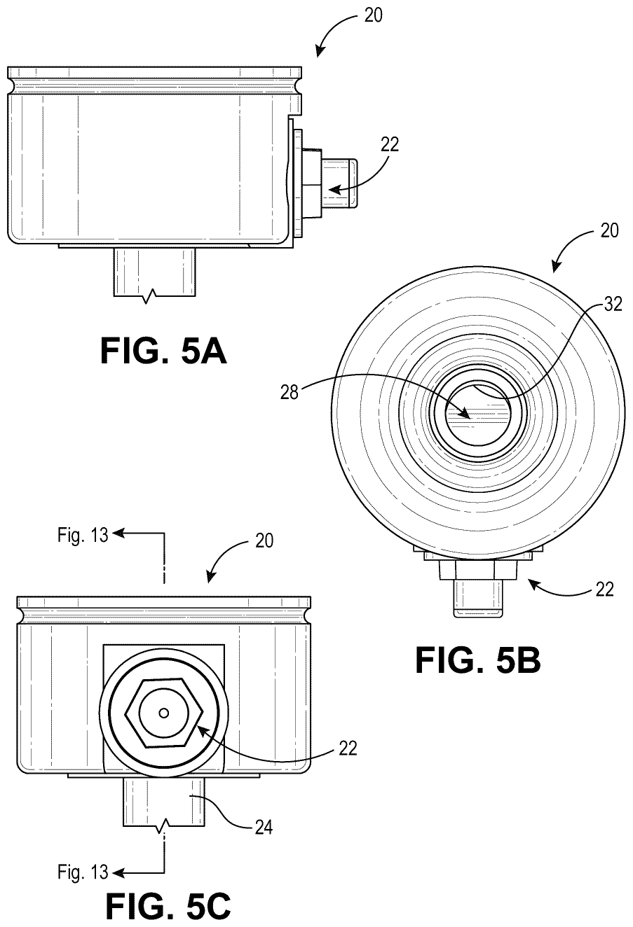

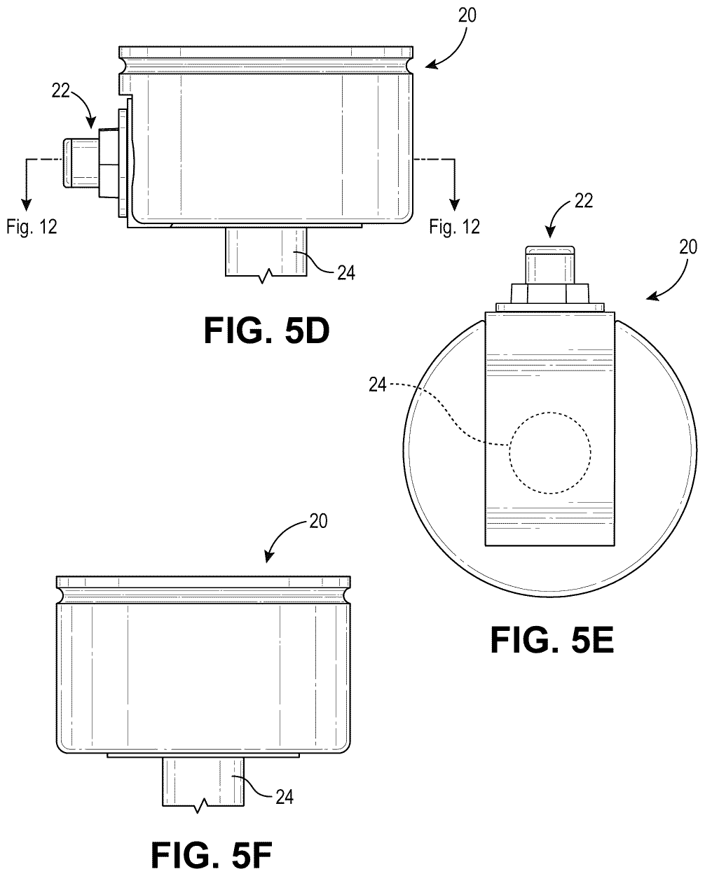

FIGS. 5A, 5B, and 5C are front, top, and left views of the lock 20, respectively. Figures D, E and F are rear, bottom and right views of the lock 20, respectively.

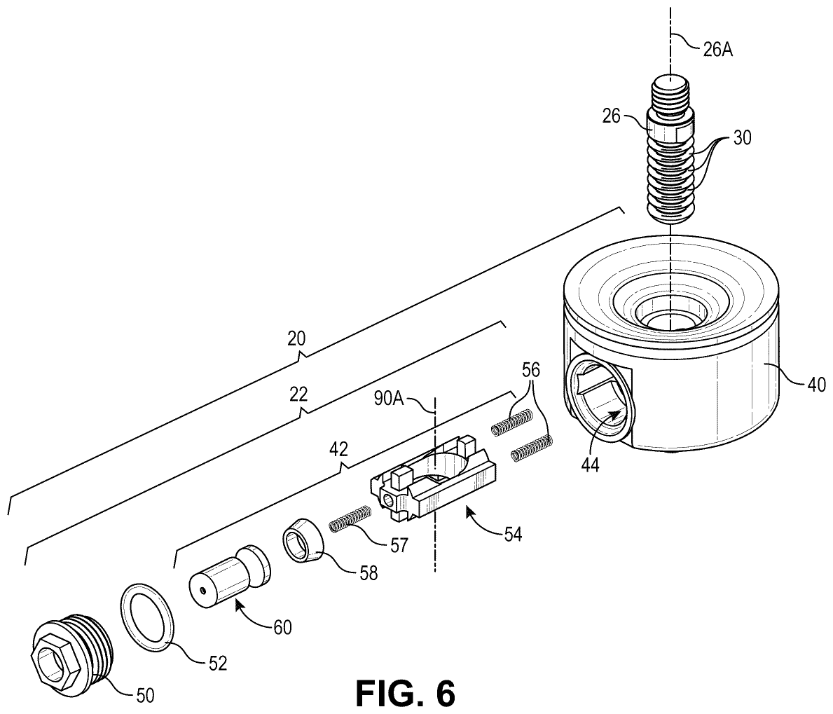

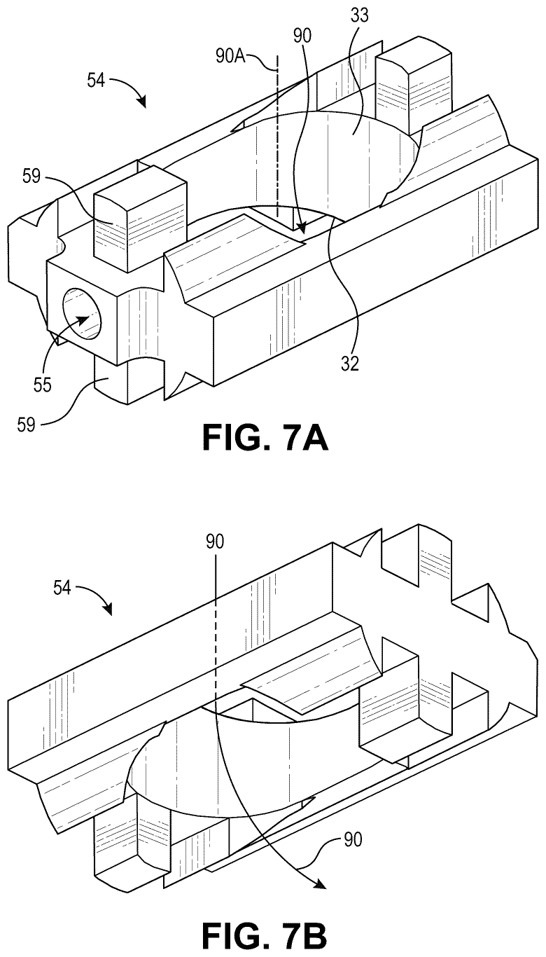

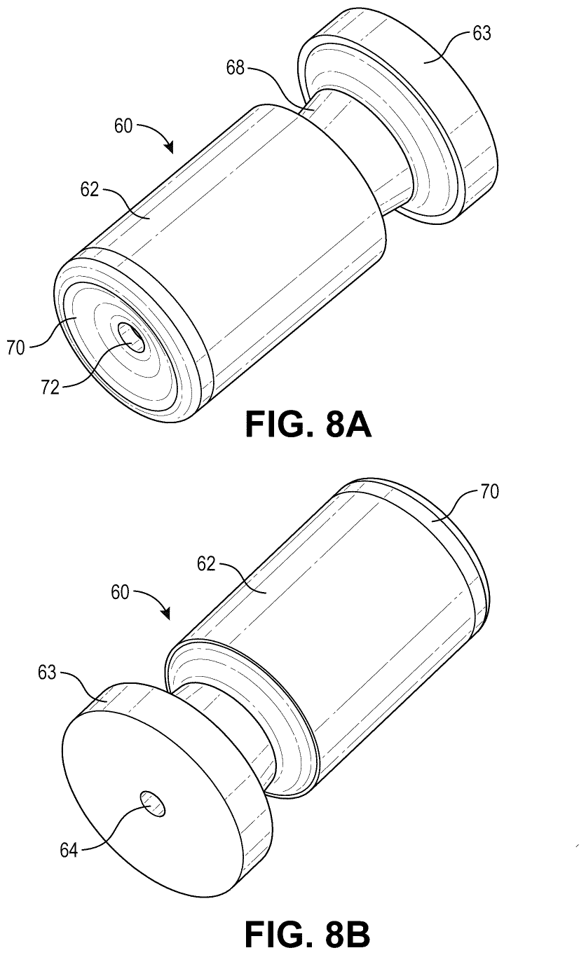

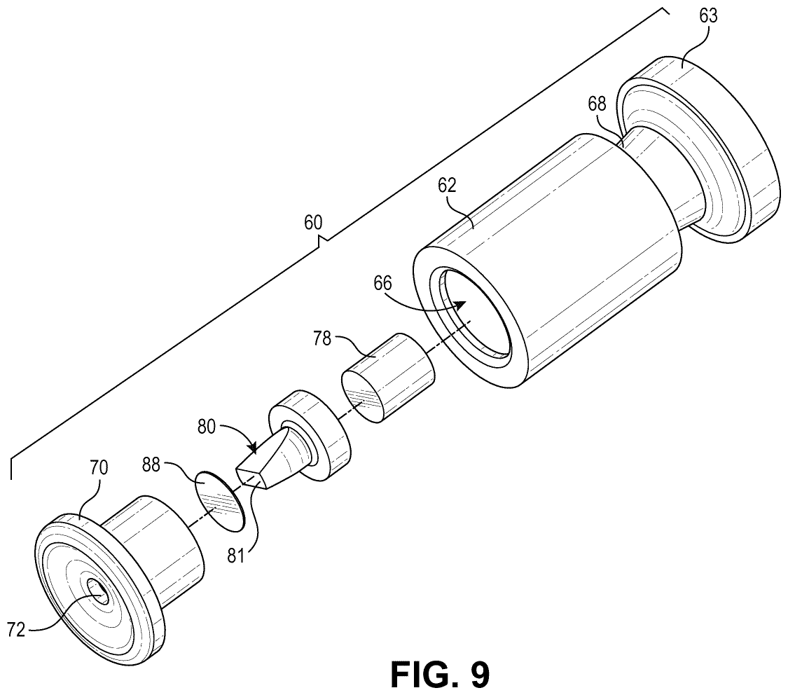

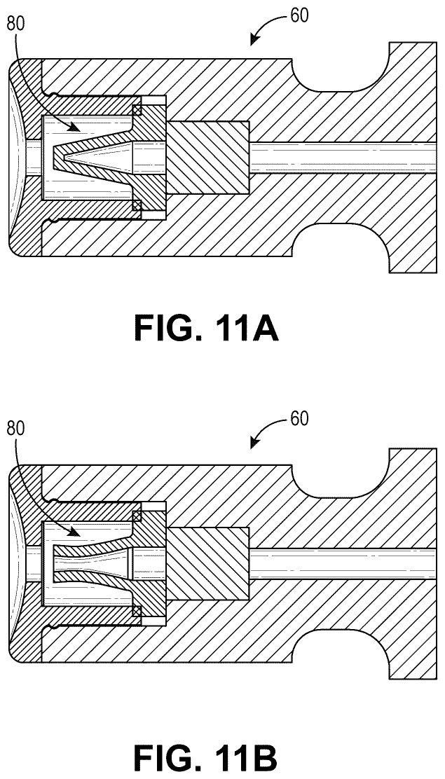

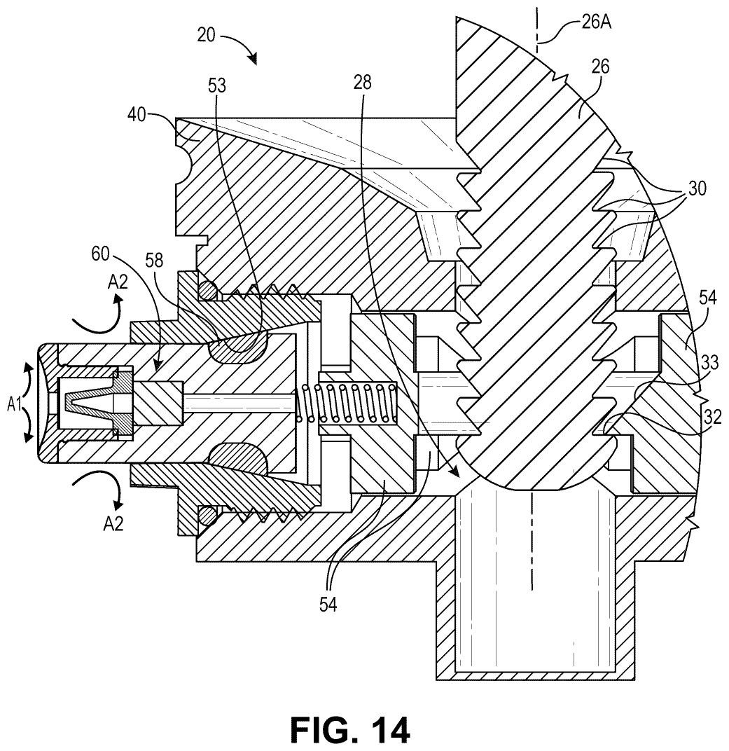

FIG. 6 is an exploded, left-rear-side perspective view of the lock 20, showing elements of the lock 20 and the liner-pin 26 having a longitudinal axis coaxial with central axial hole 28 of the lock 20. Pin 26 is detached from the liner but the pin's connection to a liner and the user of the liner will be understood by those of skill in the art. Lock 20 comprises a lock body 40 and the “push-button assembly” 22 described above. Sliding assembly 42 in FIG. 6 is a sub-set of the assembly 22 components that slide longitudinally inward and outward from and away from the body 40, for operation of the various functions described herein. The siding assembly 42 is installed in a side-aperture 44 of the body 40 and retained in the side-aperture 44 by threaded retainer 50 and its O-ring-style retainer seal 52. The sliding assembly 42 comprises a pin-lock plate 54 that is slidable inward and outward relative to the central axial hole 28 wherein the plate 54 has an aperture 90 extending through the plate 54 and having axis 90A that is parallel to the installed pin axis 26A and to the central hole 28 axis, but not always coaxial with axis 26A and the central hole axis depending on the position of the sliding plate 54. Plate 54 further has slanted surface 33 with a pin-locking edge 32 as shown in enlarged view FIGS. 7A and B, the edge being for engaging and capturing the pin 26, as understood from FIGS. 14-17. The sliding assembly further comprises inner and outer bias springs 56, 57 and expulsion valve unit 60. Springs 56 and 57, respectively, are for biasing the plate 54 outward relative to the body 40 and for biasing the expulsion valve unit 60 outward relative to the plate 54. Expulsion valve unit 60 is further detailed in FIGS. 8A and B and especially in exploded view FIG. 9, and the expulsion valve operation is shown in FIGS. 11A and B and 12-17.

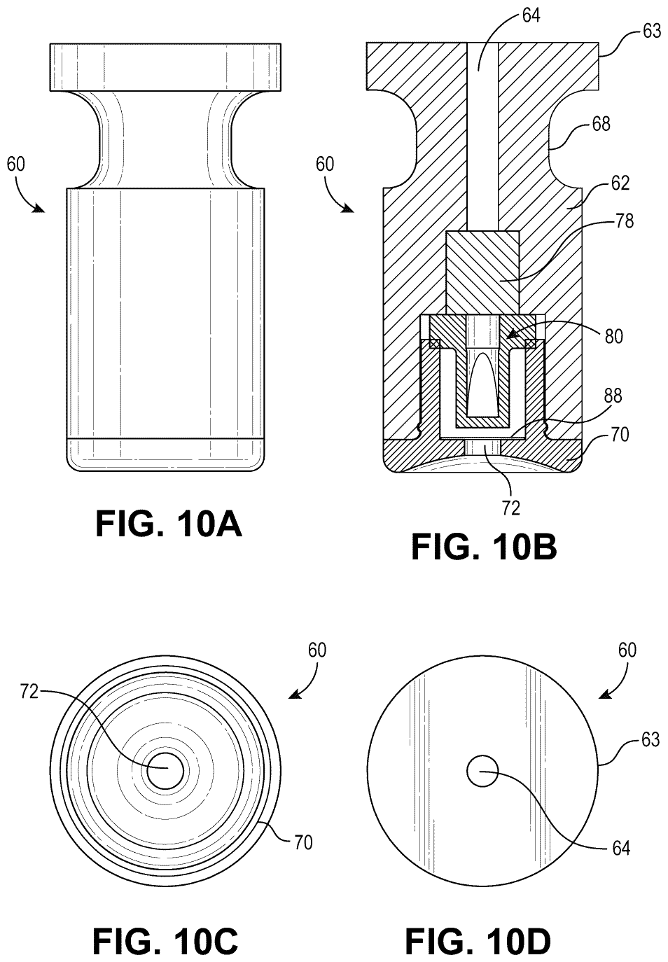

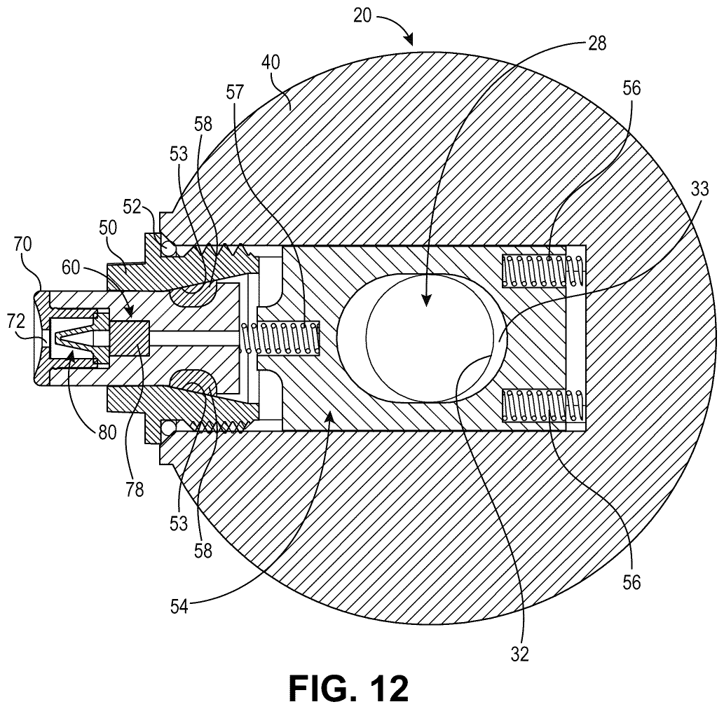

Shown in exploded FIG. 9, enlarged in FIGS. 10A-D, and the “one-way valve operation” FIGS. 11A and B, expulsion valve unit 60 may be seen to comprise hollow body 62, with an inner end 63 having air inlet 64 and an outer end having aperture 66. When the valve unit 60 is assembled, received inside the valve body 62 are foam filter 78, one-way valve 80, and adhesive mesh screen 88. Endcap 70 is connected and sealed to the outer end of valve body 62, is hollow, and has air outlet 72. This way, expelled air may flow into inner end 63, and through foam filter 78, one-way valve 80 (via its interior channel and outer end 81 when the valve 80 opens), and adhesive mesh screen 88, to exit endcap 70 at air outlet 72 to the environment. Valve body waist 68 is narrow compared to the rest of the valve body 62 including the inner end 63, and the endcap 70. Waist 68 receives suction-seal 58 that seals, when the sliding assembly 42 is in its default position (FIGS. 12-15 and 17) against the interior surface 53 of retainer 50 to create the suspension valve seal that prevents air from flowing through the lock 20 to central axial hole 28 and to socket 10, as will be further discussed below.

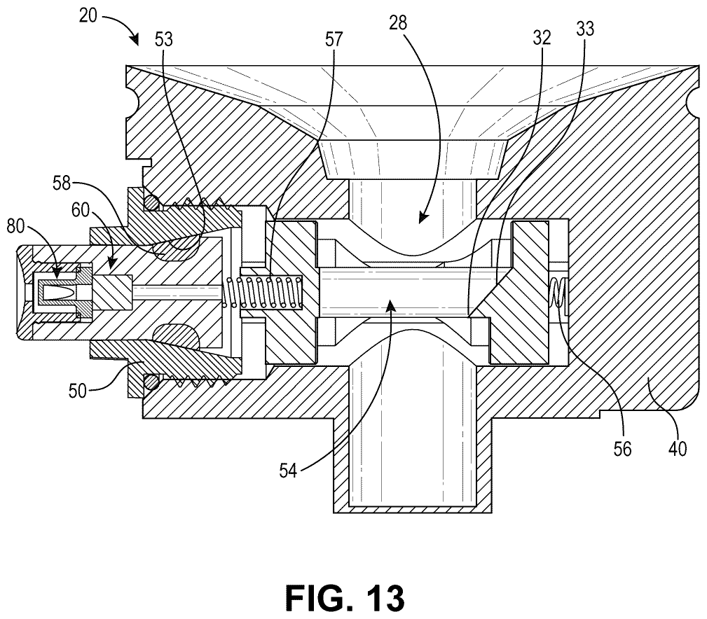

While one embodiment of the one-way valve 80 is shown in the Figures, other styles and shapes may be used. As may be understood from FIG. 9, the one-way valve embodiment 80 shown in the drawings has a broader width dimension (viewed in FIGS. 10B and 13) that is 90 degrees from a narrow width (viewed in FIGS. 11A and B, 12, 14-17), and therefore the appearance of the valve in the drawings changes depending on where the longitudinal cross-section is taken. Also, preferred expulsion valve 80 may be installed in the device in different rotational positions, while still being coaxial with the tubular body 62 and operationally effective, so that can also affect the appearance in the drawings. The elastomeric character of at least a portion of valve 80 will allow the valve to flex/bend at its normally-closed end 81 to open the air channel all the way through the valve 80 and out the endcap 70 to atmosphere (compare FIGS. 11A and 11B) in response to a higher pressure behind the valve that is higher than the valve's “crack” or “opening” pressure as discussed above in this document, thus allowing flow longitudinally through the valve.

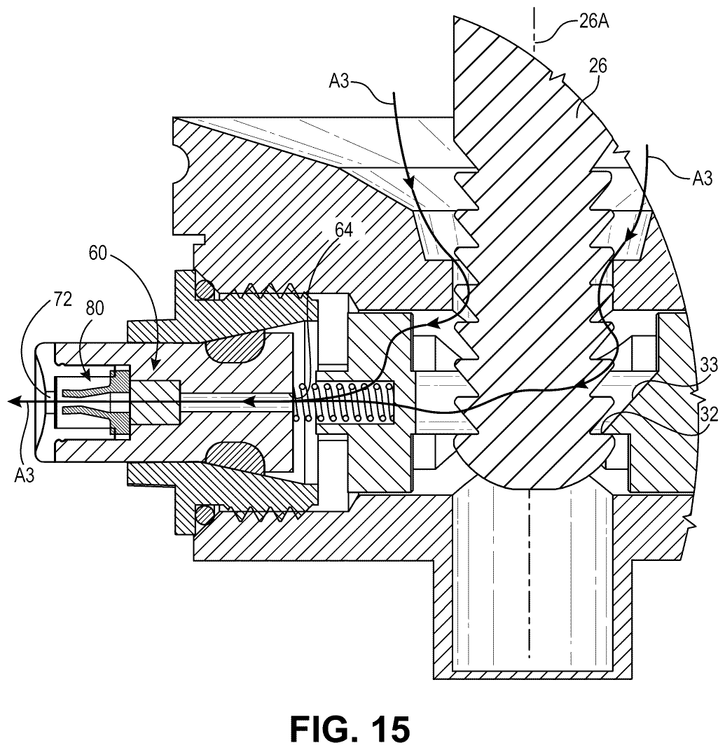

FIG. 12 portrays the lock 20 in its default condition wherein the suspension valve system is closed (seal 58 sealed against surface 53) and the expulsion valve closed, but without any pin 26 installed. Pin-locking edge 32 of the slanted surface 33, in its default position, is spring-biased to a default position wherein edge 32 protrudes slightly into the central axial hole 28, whereby, when the pin is installed in said hole 28 (as in FIGS. 14 and 15), the edge 32 extends into a thread recess 30 of the pin 26, to prevent axial movement of the pin up out of hole 28.

FIG. 13 is a cross-sectional view of the lock of FIG. 1, viewed along the line 13-13 in FIGS. 5C, with the expulsion valve and the suspension valve closed. This view shows the expulsion valve cross-sectional shape (as in FIG. 11A) resulting when the expulsion valve is cross-sectioned 90 degrees relative to FIG. 12. FIG. 13 shows edge 32 protruding to the left into the hole 28.

In all of FIGS. 12-15, and 17, the pin-locking plate 54 and the expulsion valve unit 60 are biased by springs 56, 57 toward the left in the figures. Springs 56 bias/push the plate 54 to the left, so that the plate aperture 90 extending vertically through plate 54 and having an axis 90A parallel to the longitudinal axis 26A of the pin 26 is moved slightly to the left. This way, the plate aperture axis 90A is offset slightly to the left of pin axis 26A, and pin-locking edge 32 extends into a thread-recess 30 of the pin 26 to lock the pin in the hole 28. Spring 57, between the plate 54 and the inner end of the expulsion valve body 62, biases/pushes the expulsion valve unit 60 to the left in FIGS. 11-15 and 17 as evidenced by seal 58 sealing to surface 53 FIG. 14 illustrates to best advantage the pin-locked and suspension- and expulsion-sealed positions of the elements, with pin 26 installed, for use by the wearer but before or between compressive gait portions In this scenario, retainer 50 is sealed by O-ring 52 to the lock body 40, and the suction-seal 58 is sealed against the interior surface 53 of the retainer 50, and expulsion valve 80 is closed. Thus, no atmospheric air can flow past seal 52, or flow past seal 58 between the expulsion valve body 62 and the interior surface 53, into lock body 40, so no atmospheric air can flow to the locking plate 54 and the central axis hole 28 to equalize the socket pressure with the atmospheric pressure.

The pin 26 in FIG. 14 has been fully installed, and is being held/captured by edge 32. It is noteworthy that, getting to this scenario included the pin being pushed down past the spring-biased edge 32, the edge 32 responding by moving out of the way for the threads to “ratchet down” past the edge 32, and then, upon the pin being fully lowered, the edge 32 coming to rest, biased in a recess 30. For other pins lengths or locks, the edge 32 might come to rest in a recess 30 higher up on the pin to lock the pin in the lock.

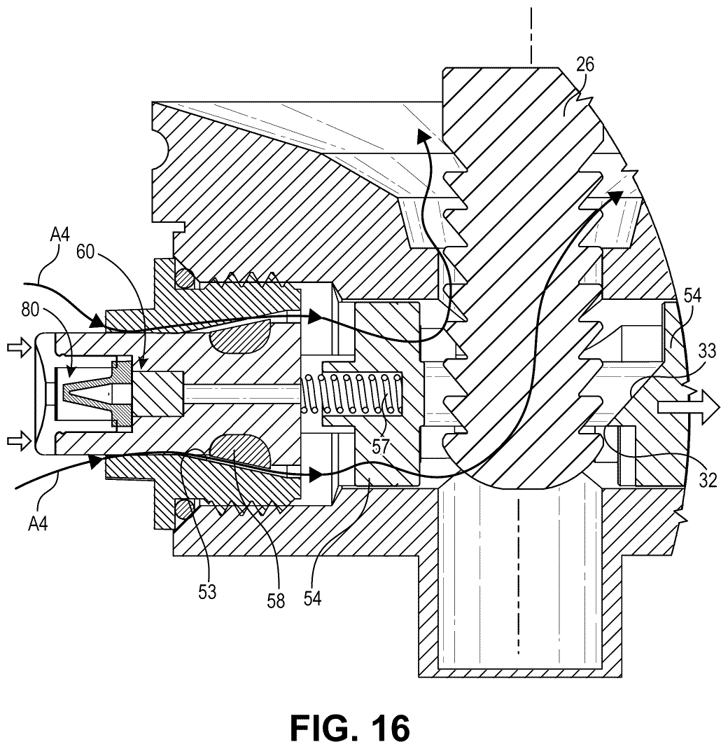

FIG. 16 illustrates to best advantage an unlocked and unsealed position of lock 20, with one-way valve 80 of the expulsion valve 80 still closed, occurring due to the wearer or other person pressing on the lock's push-button end (in this embodiment, endcap 70), as indicated by the force arrows at the far left of FIG. 16. In this position, retainer 50 is still sealed by O-ring 52 to the lock body 40, but the now-pushed multiple-functional suction control and release assembly 22, has been pushed inward by a user to move/slide the sliding assembly 42, against the bias of springs 56, 57, to the right in FIG. 16. This movement/sliding also moves the plate 54 and its locking edge 32 to the right (force/movement arrow at the far right) so that the longitudinal axis of the plate aperture axis 90A is coaxial, or close to coaxial, with the axis 26A of the pin, and the edge 32 has moved/slid out away from a thread-recess 30 of the liner pin 26 to release the pin 26 from the lock 20.

In FIG. 16, the movement of the sliding assembly 42 to the right has also unsealed seal 58 from the interior surface 53 of the retainer 50. Thus, atmospheric air can flow A4 between the expulsion valve body 62 and the interior surface 53, past seal 58, past at least portions of the pin-locking plate 54, and into the central axis hole 28 and the interior of the socket. Thus, at the same time that the pin is unlocked, the socket pressure is equalized with the atmospheric pressure, and the user may then typically easily remove the residual limb from the socket. In FIG. 16, the user has not yet raised the liner/pin up out of the lock 20 (or not yet dropped the lock and socket away from the liner/pin), but one may see that these actions are now possible because of the pin and suction release.

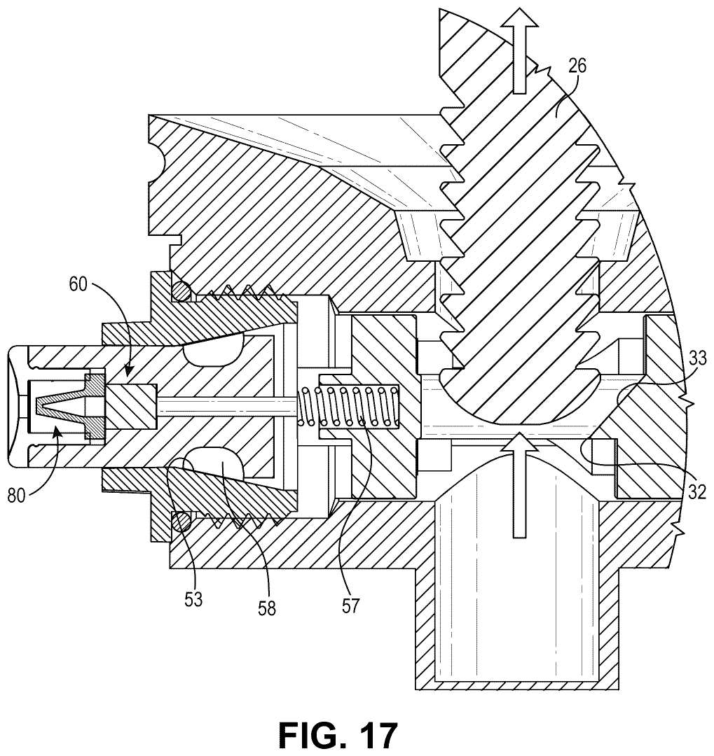

In FIG. 17, the wearer or other person has raised the pin 26 relative to the lock 20, by beginning to lift his/her residual limb and its liner out of the socket and lock (or beginning to drop the socket relative to the limb/liner), and therefore the pin is above edge 32. Further, the wearer of other person has released/stopped-pressing on the push-button end, and springs 56, 57 have biased the assembly 22 to return again to the position shown in FIG. 14, which is the default position with expulsion and suspension valve systems sealed and the edge 32 again protruding into the central axial hole 28, but in this view not contacting any pin.

It may be noted in FIGS. 7A and 12-17, that the hole 55 in the outer end of the locking plate 54 receives spring 57 and is closed at its inner end, but the “air passageway through the locking plate” (A3 in FIG. 15 and A4 in FIG. 16) may be understood to include air flowing at least around and past the posts 59 (FIG. 7A) and/or around/past/through other portions of the locking plate 54.

Typically, after pushing the push-button, airflow will be in inward A4 due to suction in the socket during typical use of the prosthetic, but outward airflow may also be possible in some circumstances, for example, if, after FIG. 17, the user keeps pushing the push-button subsequently and reinserts/repushed the residual limb into the socket 10. In such a case, air flow could flow from the socket 10 and hole 28, that is, past the locking plate 54, past seal 58, and out between the expulsion valve body 62 and the interior surface 53. Further, it will be understood from the drawings and this description that, during insertion of the liner-covered limb into the socket and lock, that air may be expelled in some circumstances through valve 80, for example, if the push-button is not pressed during insertion.

It will be understood from the Figures and this description, that the certain embodiments of the expulsion valve body 62 may be externally shaped, and cooperate with suction-seal 58, outer bias spring 57, threaded retainer 50, and retainer seal 52, in a similar way as a conventional push-button/plunger of prior art dual-suspension systems such as those available under the trademark AIRLOCK™ by Coyote O& P Inc. of Boise, Idaho. However, the invented combination of elements, features, and functions, including a nonobviously structured, placed, and operated multiple-functional suction control and release assembly, sliding assembly, and/or expulsion valve as shown and described herein, provides an effective, quiet, and convenient expulsion capability with minimal changes to the AIRLOCK™ mechanism, as certain embodiments of the one-way valve may be incorporated into the push-button/plunger without interfering with the dual-suspension pin-locking and suction-seal functions of the AIRLOCK™ system. For example, except for the air hole-apertures visible at each end of the expulsion valve unit, the expulsion valve of certain embodiments may look the same or similar to the push-button/plunger of an AIRLOCK™ system. Certain embodiments of modification of a conventional push-button/plunger may be described as hollowing-out the push-button/plunger, providing inlet 64, foam filter 78, one-way valve 80, screen 88, and outlet 72, to serve as the desired one-way valve unit 60, while maintaining the push-button/release action needed for releasing a liner pin and suction in the socket.

The lock body 40, expulsion valve housing components (body 62 and endcap 70), retainer 50, and plate 54 are preferably made from durable and rigid polymeric material(s) such as Acetal Plastic (POM). The one-way valve 80 may be made of EPDM rubber, for example. Foam filter 78 may be made of polyurethane (PU) foam, for example. Adhesive screen 88 may be made of stainless steel, for example. Seal 58 may be made of TPU/TPE rubber, for example. O-ring may be made of conventional O-ring fluid-sealing materials.

Certain embodiments, therefore, comprise, consist essentially of, or consist of: a lock at the bottom of a hard socket, the lock being adapted for connecting a limb-liner pin to a lock that is connected to a leg/post and foot prosthetic, wherein the lock also has a suspension valve that in its closed position helps maintain suction inside the socket by blocking air from flowing into the socket from the lock and the lock also has a one-way expulsion valve that opens an air pathway from the socket, through the lock, to allow air out through the lock into the environment, when the pressure rises in the socket due to air leaking into the top of the socket and threatens to seriously lessen the suction in the socket or increase the pressure in the socket above atmospheric pressure. This modulating/controlling socket pressure is done to maintain the suspension of the prosthetic limb in a pressure region chosen for each wearer that is effective for suspension and comfortable.

Certain embodiments may be described as: A suspension system for a prosthesis comprising (or consisting essentially of, or consisting of) a hard socket for receiving a residual limb wearing a limb liner, and a lock at the bottom of a hard socket, the lock being adapted to connect to post for a prosthetic foot, the lock comprising a lock body and a sliding assembly that slides inward and outward relative to the lock body, the sliding assembly comprising a plate adapted to secure a pin of the liner into a central hole of the lock when the sliding assembly is slid outward, and the sliding assembly also comprising a suspension valve seal that seals against a surface of the lock, when the sliding assembly is slid outward, to prevent air from flowing past said seal, into the lock, and into an interior space of the hard socket between a bottom end of the liner and an interior surface at the bottom of the hard socket; the sliding assembly further comprising a one-way expulsion valve having an inward end in fluid communication with the central hole of the lock and the interior space of the hard socket, and having an outer end in fluid communication with atmosphere around the lock, wherein the expulsion valve opens, when the sliding assembly is slid outward, when pressure in the interior space is above a crack pressure of the expulsion valve, to expel air from the interior space to said atmosphere around the lock. The suspension system above may further be described as a system wherein the expulsion valve is inside an expulsion valve body that has an outer end that is the outermost extremity of the sliding assembly, so the outer end is pushed to actuate said sliding inward, and the seal is located on an outer surface of the expulsion valve body; and/or where the plate has an aperture defined by a surface comprising an edge protruding into the aperture, and said edge extends into a recess between threads of the pin to secure the pin in the lock; and/or wherein said surface of the lock against which the suspension valve seal seals is an interior surface of a retainer that connects to the lock body and holds the sliding assembly slidably in the lock, the retainer having a central aperture through which the expulsion body extends so that the outer end of the expulsion body is accessible for being pushed to slide the sliding assembly inward. The suspension system described above may include the expulsion valve having a crack pressure of 1-3 psi above atmospheric pressure around the lock. In certain embodiments, the system does not comprise a vacuum pump. In certain embodiments, the system does not comprise a lanyard strap for connecting the liner to the hard socket.

Certain embodiments may be described as: A suspension system for a prosthesis to be attached to a residual limb, the system comprising a hard socket with a lock having a lock body with a central axial hole receiving a pin of a limb-liner, and sliding assembly adapted to slide inward and outward relative to a body of the lock and adapted to mechanically capture the pin of the limb-liner to prevent the pin from being lifted out of the lock, the sliding assembly further comprising a seal adapted to seal against a surface of the lock when the sliding assembly is slid outward and to unseal from the surface of the lock to open an air passage through the lock when the sliding assembly slides inward relative to the body, and the sliding assembly further comprising a one-way expulsion valve, with a crack pressure, for releasing air flowing from the central axial hole, through the lock, and to atmosphere around the lock, when pressure in the interior space builds up above the crack pressure, to modulate socket pressure to maintain the suspension of the prosthetic limb in a pressure region chosen for each wearer that is effective for suspension and comfortable. Certain of the systems described above includes the expulsion valve having a crack pressure in the range of 1-3 psi (or in some cases in the range of up to 5 psi) above atmospheric pressure around the lock. Certain of the systems described above do not comprise a vacuum pump. Certain of the systems described above do not comprise a lanyard strap for connecting the liner to the hard socket.

Although this disclosed technology has been described above with reference to particular means, materials, and embodiments, it is to be understood that the disclosed technology is not limited to these disclosed particulars but extends instead to all equivalents within the broad scope of this disclosure including the drawings and the claims.

1. A suspension system for a prosthesis comprising a hard socket for receiving a residual limb wearing a limb liner, and a lock at the bottom of a hard socket, the lock being adapted to connect to post for a prosthetic foot, the lock comprising a lock body and a sliding assembly that slides inward and outward relative to the lock body, the sliding assembly comprising a plate adapted to secure a pin of the liner into a central hole of the lock when the sliding assembly is slid outward, and the sliding assembly also comprising a suspension valve seal that seals against a surface of the lock, when the sliding assembly is slid outward, to prevent air from flowing past said seal, into the lock, and into an interior space of the hard socket between a bottom end of the liner and an interior surface at the bottom of the hard socket;
the sliding assembly further comprising a one-way expulsion valve having an inward end in fluid communication with the central hole of the lock and the interior space of the hard socket, and having an outer end in fluid communication with atmosphere around the lock, wherein the expulsion valve opens, when the sliding assembly is slid outward, when pressure in the interior space is above a crack pressure of the expulsion valve, to expel air from the interior space to said atmosphere around the lock.

2. The suspension system as in claim 1, wherein the expulsion valve is inside an expulsion valve body that has an outer end that is the outermost extremity of the sliding assembly, so the outer end is pushed to actuate said sliding inward, and the seal is located on an outer surface of the expulsion valve body.

3. The suspension system as in claim 1, where the plate has an aperture defined by a surface comprising an edge protruding into the aperture, and said edge extends into a recess between threads of the pin to secure the pin in the lock.

4. The suspension system as in claim 2, wherein said surface of the lock against which the suspension valve seal seals is an interior surface of a retainer that connects to the lock body and holds the sliding assembly slidably in the lock, the retainer having a central aperture through which the expulsion body extends so that the outer end of the expulsion body is accessible for being pushed to slide the sliding assembly inward.

5. The suspension system of claim 1 wherein the expulsion valve has a crack pressure of 1-3 psi above atmospheric pressure around the lock.

6. The suspension system of claim 1 that does not comprise a vacuum pump.

7. The suspension system of claim 1 that does not comprise a lanyard strap for connecting the liner to the hard socket.

8. A suspension system for a prosthesis to be attached to a residual limb, the system comprising a hard socket with a lock having a lock body with a central axial hole receiving a pin of a limb-liner, and sliding assembly adapted to slide inward and outward relative to a body of the lock and adapted to mechanically capture the pin of the limb-liner to prevent the pin from being lifted out of the lock, the sliding assembly further comprising a seal adapted to seal against a surface of the lock when the sliding assembly is slid outward and to unseal from the surface of the lock to open an air passage through the lock when the sliding assembly slides inward relative to the body, and the sliding assembly further comprising a one-way expulsion valve, with a crack pressure, for releasing air flowing from the central axial hole, through the lock, and to atmosphere around the lock, when pressure in the interior space builds up above the crack pressure, to modulate socket pressure to maintain the suspension of the prosthetic limb in a pressure region chosen for each wearer that is effective for suspension and comfortable.

9. The suspension system of claim 8 wherein the expulsion valve has a crack pressure of 1-3 psi above atmospheric pressure around the lock.

10. The suspension system of claim 8 that does not comprise a vacuum pump.

11. The suspension system of claim 8 that does not comprise a lanyard strap for connecting the liner to the hard socket.
